(19) United States
(12) Patent Application Publication
(10) Pub. No.: US 2003/0105143 A1
(43) Pub. Date: Jun. 5, 2003

(54) Selective antibacterial agents

(76) Inventors: Aldo Ammendola, Munchen (DE); Bernd Kramer, Aachen (DE); Wael Saeb, Pinnegg Martinsried (DE)

Correspondence Address: Carol A. Schneider, IRELL & MANELLA LLP, Suite 900, 1800 Avenue of the Stars, Los Angeles, CA, 90067, US

(21) Appl. No.: 10/094,301

(22) Filed: Mar. 8, 2002

Related U.S. Application Data
(63) Continuation of application No. PCT/EP01/012875, filed on Nov. 7, 2001.

(51) Int. Cl. A61K 31/44; A61K 31/40; A61K 31/137; A61K 31/13

(52) U.S. Cl. 514/357; 514/649; 514/408; 514/674

(57) ABSTRACT

The present invention relates to the use of compounds of the general Formula (I)

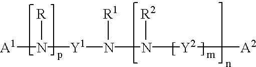

for the regulation of the quorum sensing system of microorganisms wherein in Formula (I),

R is H, alkyl, cycloalkyl, aryl or heteroaryl;

R¹ is H, alkyl, cycloalkyl, aryl or heteroaryl;

R² is H, alkyl, cycloalkyl, aryl or heteroaryl;

A¹ and A² each independently represent an optionally substituted C₁-C₂₀-alkyl group which may contain one or more group(s) Z, or a mono cyclic or polycyclic optionally substituted aromatic or non-aromatic ring system which may contain one or more group(s) X, and in case of a polycyclic ring system, said system contains at least one aromatic ring;

Z is selected from the group consisting of S, O, N, NR⁴, CO, CO₂, CS, SO or SO₂

X is selected from the group consisting of S, O, N, NR⁴, SO or SO₂;

said substituted ring system carries a substituent R³ on one or more of the carbon atoms of said ring system;

said substituted C₁-C₂₀-alkyl group carries a substituent R³ on one or more of the carbon atoms of said alkyl group;

R³ is independently H, OR⁴, SR⁴, hydroxyalkyl, hydroxyalkylamino, cycloalkyl, halogen, haloalkyl, haloalkyloxy, NO₂, CN, SO₂NR⁴R⁵, CO₂NR⁴R⁵, COR⁴, CO₂R⁴, SO₂R⁴, SO₃R⁴, NR⁴R⁵, alkyl, aryl or heteroaryl;

R⁴ is H, alkyl, cycloalkyl, aryl or heteroaryl;

R⁵ is H, O-alkyl, O-aryl, alkyl, heteroaryl or aryl;

Y¹ and Y² are independent from each other C═O, C═S, SO₂ or C═NR⁵;

p is 0, m is 0, n is 0;

or p is 0, m is 1, n is 1;

or p is 1, m is 0, n is 1;

or p is 0, m is 0, n is 1;

or p is 1, m is 0, n is 0;

or p is 1, m is 1, n is 1.

The present invention relates to the use of compounds such as amide, carbazide, hydrazide, urea, and guanidine derivatives as selective inhibitors of bacterial pathogens. In particular the invention refers to a family of compounds that block the quorum sensing system of Gram-negative bacteria, a process for their manufacture, pharmaceutical compositions containing them and to their use for the treatment and prevention of microbial damages and diseases, in particular for diseases where there is an advantage in inhibiting quorum sensing regulated phenotypes of pathogens.

Many microorganisms, including bacteria, fungi, protozoa and algae cause severe damages or diseases in different areas such as industry, agriculture, environment and medicine. Especially bacteria as human pathogens cause tremendous costs in public health systems worldwide. The continuing emergence of multiple-drug-resistant bacterial strains has necessitated finding new compounds that can be used in antibacterial treatment. There are two broad strategies for the control of bacterial infection: either to kill the organism or to attenuate its virulence such that it fails to adapt to the host environment. The latter approach has, however, lacked specific targets for rational drug design. The discovery that Gram-negative bacteria employ a signal transduction pathway comprising a small molecule to globally regulate the production of virulence determinants offers such a novel target.

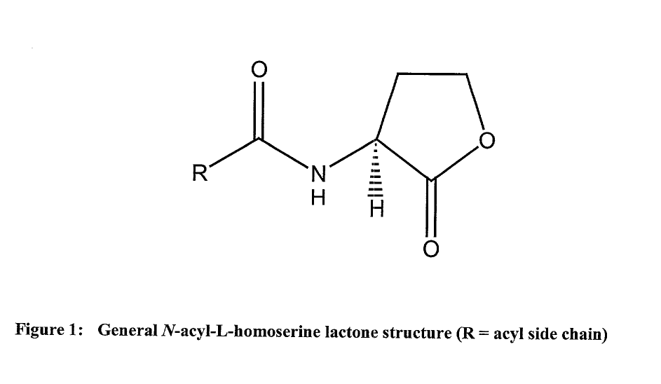

A wide variety of Gram-negative bacteria produce N-acyl-L-homoserine lactone (AHL or HSL, FIG. 1) derivatives as signal molecules in intercellular communication. These molecules, also referred to as “pheromones” or “quoromones”, comprise a homoserine lactone moiety linked to an acyl side chain. Bacteria use this signaling system to monitor their population cell density in a process referred to as “quorum sensing”. In each cell of a population an HSL synthase from usually the LuxI family of proteins produce a low basal level of diffusible HSLs. The HSL concentration increases with bacterial population density until a threshold concentration is reached which results in expression of various HSL-dependent genes through an HSL-receptor protein belonging generally to the LuxR family of transcriptional regulators. This HSL-receptor protein complex serves not only as positive transcription regulator of quorum sensing regulated genes but also as positive regulator for the HSL synthesis itself. Therefore, the entire system is amplified via a process of autoinduction.

This system was first discovered in the bioluminescent marine bacteria Vibrio harveyi and V. fischeri where it is used to control bioluminescence expression. In recent years it has become apparent that many Gram-negative bacteria employ one or more quorum sensing systems comprising HSL derivatives with different acyl side chains to regulate in a cell-density dependent manner a wide variety of physiological processes such as swarming motility, biofilm formation, pathogenicity, conjugation, bioluminescence or production of pigments and antibiotics (Table 1, for reviews and further references see, e.g.: Fuqua et al., Ann. Rev. Microbiol. 50:727-51, 1996; Fuqua & Greenberg, Curr. Opinion Microbiol. 1:183-89, 1998; Eberl, Syst. Appl. Microbiol. 22:493-506, 1999; De Kievit & Iglewski, Infect. Immun. 68:4839-49, 2000).

TABLE 1
Summary of HSL-based quorum sensing systems

| | Regulatory | | |
|---|---|---|---|
| Bacterium | proteins | Major HSL | HSL-regulated phenotype |
| Aeromonas hydrophila | AhyR, AhyI | C4-HSL | Extracellular protease, |
| | | | biofilm formation |
| Aeromonas salmonicida | AsaR, AsaI | C4-HSL | Extracellular protease |
| Agrobacterium tumefaciens | TraR, TraI | 3-oxo-C8-HSL | Conjugal transfer |
| Burkholderia cepacia | CepR, CepI | C8-HSL | Protease, lipase, ornibactin |
| | | | synthesis, biofilm formation, |
| | | | swarming motility |
| Chromobacterium violaceum | CviR, CviI | C6-HSL | Antibiotics, violacein, |
| | | | exoenzymes, cyanide |
| Enterobacter agglomerans | EagR, EagI | 3-oxo-C6-HSL | Unknown |
| Erwinia carotovora | CarR, (CarI) | 3-oxo-C6-HSL | Carbapenem antibiotics, |
| | ExpR, ExpI | | exoenzyme production |
| Erwinia chrysanthemi | ExpR, ExpI | 3-oxo-C6-HSL | Pectinase expression |
| | (EchR, EchI) | | |
| Escherichia coli | SdiA | Unknown | Cell division, virulence |
| | | | factor production |
| Nitrosomonas europaea | Unknown | 3-oxo-C6-HSL | Emergence from lag phase |
| Obesumbacterium proteus | OprR, OprI | 3-oxo-C6-HSL | Unknown |
| Pantoea stewartii | EsaR, EsaI | 3-oxo-C6-HSL | Exopolysaccharide |
| | | | production, virulence factor |
| | | | production |
| Pseudomonas aeruginosa | LasR, LasI | 3-oxo-C12- | Extracellular virulence |
| | | HSL | factors, Xcp, biofilm |
| | | | formation, RpoS, RhlR |
| Pseudomonas aeruginosa | Rh1R, Rh1I | C4-HSL | Extracellular virulence |
| | | | factors, cyanide, lectins, |
| | | | pyocyanin, rhamnolipid, type |
| | | | 4 pili, twitching motility |
| Pseudomonas aureofaciens | PhzR, PhzI | C6-HSL | Phenazine antibiotics |
| Pseudomonas fluorescens | HdtS | 3-hydroxy-7- | Unknown |
| | | cis-C14-HSL | |
| Ralstonia solanacearum | So1R, So1I | C8-HSL | Unknown |
| Rhizobium etli | RaiR, RaiI | 7 HSLs | Root nodulation |
| Rhizobium leguminosarum | RhiR | 3-hydroxy-7- | Nodulation, bacteriocin, |
| | | cis-C14-HSL | stationary phase survival |
| Rhizobium leguminosarum | RhiR, RhiI | C6-HSL, | rhizome interactions |
| | | C8-HSL | |
| Rhodobacter sphaeroides | CerR, CerI | 7-cis-C14-HSL | Clumping factor |
| Serratia liquefaciens | SwrR, SwrI | C4-HSL | Swarming motility, protease, |
| | | | serrawettin W2, lipase |
| Vibrio anguillarum | VanR, VanI | 3-oxo-C10- | Unknown |
| | | HSL | |
| Vibrio anguillarum | VanM, | C6-HSL, | Unknown |
| | VanN | 3-.hydroxy-C6- | |
| | | HSL | |
| Vibrio fischeri | LuxR, LuxI | 3-oxo-C6-HSL | Bioluminescence |
| Vibrio harveyi | LuxM, | 3-hydroxy-C4- | Bioluminescence, PHB |
| | LuxN | HSL | synthesis |
| Xenorhabdus nematophilus | Unknown | 3-hydroxy-C4- | Virulence |
| | | HSL | |
| Yersinia enterocolitica | YenR, YenI | C6-HSL, | Unknown |
| | | 3-oxo-C6-HSL | |
| Yersinia pestis | YpeR, YpeJ | Unknown | Unknown |
| Yersinia pseudotuberculosis | YpsR, YpsI | 3-oxo-C6-HSL | Motility, clumping |
| Yersinia pseudotuberculosis | YtbR, YtbI | C8-HSL | Unknown |
| Yersinia ruckeri | YukR, YukI | Unknown | Unknown |

With regard to bacteria that utilize HSL-based quorum sensing as part of their lifestyle, Pseudomonas aeruginosa is perhaps the best understood in terms of the role quorum sensing plays in pathogenicity. In this human opportunistic pathogen, which causes nosocomial infections in immunocompromized patients and has an extremely high potential to develop resistance mechanisms against traditional antibiotic treatment, production of many virulence factors including several proteases, exotoxin A, rhamnolipid, pyocyanin, cyanide and chitinase is regulated by two interlinked quorum sensing circuits. Moreover, it has been demonstrated that this signaling system is involved in the ability of P. aeruginosa to form biofilms (Davies et al., Science 280:295-8, 1998). Recently Huber et al. (Microbiology 147:2517-28, 2001) demonstrated that biofilm formation and swarming motility of Burkholderia cepacia, like P. aeruginosa a human opportunistic pathogen, is also dependent on an HSL-based quorum sensing system.

Biofilms are defined as an association of microorganisms growing attached to a surface and producing a slime layer of extracellular polymers in which the microbial consortia is embedded in a protective environment (for a review see: Costerton et al., Ann. Rev. Microbiol. 49:711-45, 1995). Biofilms represent a severe problem as bacteria integrated in such a polymer matrix develop resistance to conventional antimicrobial agents. P. aeruginosa cells, for example, growing in an alginate slime matrix have been demonstrated to be resistant to antibiotics (e.g., aminoglycosides, β-lactam antibiotics, fluoroquinolones) and disinfectants (Govan & Deretic, Microbiol. Rev. 60:539-74, 1996). Several mechanisms for biofilm-mediated resistance development have been proposed (Costerton et al., Science 284:1318-22, 1999).

In most natural, clinical and industrial settings bacteria are predominantly found in biofilms. Drinking water pipes, ship hulls, teeth or medical devices represent typical surfaces colonized by bacteria. On the one hand biofilms decrease the life time of materials through corrosive action in the industrial field, a process also referred to as “biofouling”. Furthermore, microbial biofilms growing for example on ship hulls increase fuel consumption through enhanced frictional resistance and simultaneously reduce maneuverability. On the other hand two thirds of all bacterial infections in humans are associated with biofilms (Lewis, Antimicrob. Agents Chemother. 45:999-1007, 2001).

Pseudomonas aeruginosa, for example, forms infectious biofilms on surfaces as diverse as cystic fibrosis lung tissue, contact lenses, and catheter tubes (Stickler et al., Appl. Environm. Microbiol. 64:3486-90, 1998). Burkholderia cepacia also forms biofilms in lungs of cystic fibrosis patients and is a major industrial contaminant (Govan et al., J. Med. Microbiol. 45:395-407, 1996). Since biofilm formation of both organisms is demonstrated to require an HSL signaling system, inhibition of their quorum sensing systems would result in an impaired ability to form biofilms and therefore in an increased susceptability to antibacterial treatment.

The discovery that a wide spectrum of organisms use quorum sensing to control virulence factor production and other phenotypes such as biofilm formation makes it an attractive target for antimicrobial therapy. Pathogenic organisms using this signaling system to control virulence could potentially be rendered avirulent by blocking this cell-cell communication system. In contrast to traditional antibiotics, the risk of resistance development seems to be very low, since quorum sensing blocking agents would not kill the organism but disturb signal transduction pathways. There are several possibilities of interrupting the quorum sensing circuit.

For example, plants expressing an HSL-lactonase enzyme originally derived from Bacillus sp. have been demonstrated to quench pathogen quorum sensing signaling and to significantly enhance resistance to Erwinia carotovora infections (Dong et al., Nature 411:813-7, 2001). An alternative way to block cell signaling could be to interrupt the HSL synthesis by using analogs of HSL precursors.

However, the most promising possibility to block quorum sensing is to take advantage of the unique specificity the HSLs and HSL-receptor proteins show for one another. The ability of homoserine lactone-based analogs to inhibit activation of HSL-receptor proteins has already been demonstrated in a number of bacteria including Vibrio fischeri (Schaefer et al., J. Bacteriol. 178:2897-901, 1996), Agrobacterium tumefaciens (Zhu et al., J. Bacteriol. 180:5398-405, 1998), Chromobacterium violaceum (McLean et al., Microbiology 143:3703-11, 1997), Aeromonas salmonicida (Swift et al., J. Bacteriol. 179:5271-81, 1997) and Pseudomonas aeruginosa (Pesci et al., J. Bacteriol. 179:3127-32, 1997). However, none of these compounds have been developed as antimicrobial agents, e.g. in medical therapy, so far.

The only described non-HSL-based antimicrobials which are supposed to interfere specifically with HSL-regulated processes are halogenated furanone derivatives which are structurally similar to HSLs and have been isolated from red marine algae Delisea pulchra (WO 96/29392). Additionally, these substances have been demonstrated to inhibit also Gram-positive bacteria (WO 99/53915). However, the use of most of these compounds is limited due to their toxicity making them unsuitable for veterinary and medical applications.

Many target genes involved in biofilm formation, methods of screening for compounds to control biofilm development and HSL-based compositions to prevent biofilm formation have been described (WO 99/55368, WO 98/57618, WO 99/27786, WO 98/58075), but until now no promising antibacterial drug candidate has been developed that is capable of inhibiting biofilm formation in different areas, preferentially in the medical field.

It is an object of the present invention to provide compounds blocking specifically quorum sensing regulated processes without inhibiting bacterial growth. Furthermore, these compounds should not be structural derivatives of the homoserine lactone family of regulatory compounds and should not exhibit any toxic properties.

Accordingly, we have been able to find compounds that can significantly reduce virulence gene expression and biofilm formation of several human pathogens. In contrast to the furanones the compounds of this invention do not show any toxic effect and are therefore suitable for applications in a wide area. Such applications could be the use of the compounds for instance as new antibiotic therapeutics, disinfectants, antifouling coatings or coatings of medical devices. In contrast to traditional antibacterial agents (like amide or 1,2-acylhydrazine derivatives in WO 01/51456; for the synthesis of amide or 1,2-acylhydrazine derivatives see also EP 638545 and EP 982292), the compounds of the present invention do not kill the microorganisms, but render them avirulent. The advantage of this alternative strategy is that the emergence of bacterial resistance against such antimicrobials is extremely improbable.

In general, the present invention provides compounds selectively modulating bacterial cell-cell communication. Through inhibition of this communication system the expression of many HSL-dependent virulence genes and other phenotypes like swarming motility and biofilm formation are significantly reduced or completely abolished rendering a bacterial population more susceptible to the host immune,-response or to treatment with traditional antibacterial agents.

Thus, in one aspect, the invention refers to a method for inhibiting an HSL-regulated process in a microorganism by exposing the microorganism to a new class of compounds with an inhibitory effect on bacterial signaling.

The present invention therefore refers to compounds of the general Formula (I)

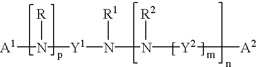

wherein

R is H, alkyl, cycloalkyl, aryl or heteroaryl;

R¹ is H, alkyl, cycloalkyl, aryl or heteroaryl;

R² is H, alkyl, cycloalkyl, aryl or heteroaryl;

A¹ and A² each independently represent an optionally substituted C₁-C₂₀-alkyl group which may contain one or more group(s) Z, or a monocyclic or polycyclic optionally substituted aromatic or non-aromatic ring system which may contain one or more group(s) X, and in case of a polycyclic ring system, said system contains at least one aromatic ring;

Z is selected from the group consisting of S, O, N, NR⁴, CO, CO₂, CS, SO or SO₂

X is selected from the group consisting of S, O, N, NR⁴, SO or SO₂;

said substituted ring system carries a substituent R³ on one or more of the carbon atoms of said ring system;

said substituted C₁-C₂₀-alkyl group carries a substituent R³ on one or more of the carbon atoms of said alkyl group;

R³ is independently H, OR⁴, SR⁴, hydroxyalkyl, hydroxyalkylamino, cycloalkyl, halogen, haloalkyl, haloalkyloxy, NO₂, CN, SO₂NR⁴R⁵, CO₂NR⁴R⁵, COR⁴, CO₂R⁴, SO₂R⁴, SO₃R⁴, NR⁴R⁵, alkyl, aryl or heteroaryl;

R⁴ is H, alkyl, cycloalkyl, aryl or heteroaryl;

R⁵ is H, O-alkyl, O-aryl, alkyl, heteroaryl or aryl;

Y¹ and Y² are independent from each other C═O, C═S, SO₂ or C═NR⁵;

p is 0,m is 0, n is 0;

or p is 0, m is 0, n is 1;

or p is 0, m is 1, n is 1;

or p is 1, m is 0, n is 0;

or p is 1, m is 0, n is 1;

or p is 1, m is 1, n is 1;

The invention also provides a pharmaceutical composition comprising a compound of Formula (I), in free form or in the form of pharmaceutically acceptable salts and physiologically functional derivatives, together with a pharmaceutically acceptable diluent or carrier therefore.

The term “physiologically functional derivative” as used herein refers to compounds which are not pharmaceutically active themselves but which are transformed into their pharmaceutical active form in vivo, i.e. in the subject to which the compound is administered.

In another aspect, the present invention also provides a method for the treatment or prophylaxis of a condition where there is an advantage in inhibiting quorum sensing which comprises the administration of an effective amount of a compound of Formula (I) and physiologically acceptable salts or physiologically functional derivatives thereof. The term “quorum sensing” is intended to describe cell-density dependent gene regulation through a diffusible signal molecule (Fuqua et al., J. Bacteriol. 176:269-75, 1994).

The invention is also directed to the use of compounds of Formula (I) and of their pharmacologically tolerable salts or physiologically functional derivatives for the production of a medicament or medical device for the prevention and treatment of diseases, where quorum sensing inhibition is beneficial. Furthermore, the invention is also directed to the use of compounds of Formula (I) and of their pharmacologically tolerable salts or physiologically functional derivatives for the production of an antibacterial agent for the prevention and treatment of bacterial biofilms in industrial and environmental settings.

In addition, the present invention provides methods for preparing the desired compounds of Formula (I).

One possibility for the synthesis of compounds of Formula (I) (m, n, p=0) comprises the step of reacting an amine of Formula (II) with a compound of Formula (III). Possibilities for preparing different amides are described by J. Zabicky in “The Chemistry of Amides”, in the serial of S. Patai (ed.), “The Chemistry of Functional Groups”, John Wiley & Sons, 1975, p. 74-131. Methods for preparing thioamides are described in Houben-Weyl, J. Falbe (ed.), G. Thieme Verlag, vol. E5, p. 1219-59. Methods for preparing sulfamides are described by Caldwell et al., J. Am. Chem. Soc. 1944, 66, 1479-82, or by Flynn et al., Med. Chem. Res., 1998, 8, 219-43 and Dziadulewicz et al., Bioorg. Med. Chem. Lett. 2001, 11, 5, 705-10.

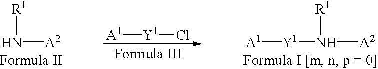

One method for preparing the compounds of Formula (I) (p=0 / m, n=1) comprises the step of reacting a compound of Formula (IV) with a compound of Formula (III). Other methods for preparing different 1,2-diacylhydrazines are described in Houben-Weyl, “Methoden der organischen Chemie”, Vierte Auflage, G. Thieme Verlag, J. Falbe (ed.), vol. E5, p. 1173-80 or P. A. S. Smith, “Open-Chain Organic Nitrogen Compounds”, W. A. Benjamin Inc., New York, vol. 2, p. 173-201. Methods for preparing different 1,2-disulfonylhydrazines are described in Arch. Pharm. 1953, 286, 338-43 or in U.S. Pat. No. 6,291,504. Methods for preparing 1-acyl-2-sulfonylhydrazines are described in Russ. J. Gen. Chem. 2000, 70, 3, 459-60 or by Leadini et al., J. Chem. Soc. Perkin Trans. 1 1998, 1833-8 and by M. Reinecke et al., J. Org. Chem. 1988, 53, 1, 208-10.

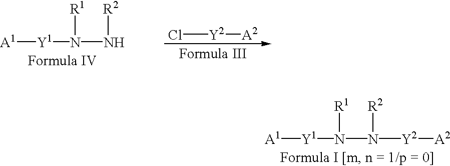

One possibility for the synthesis of compounds of Formula (I) (m, n, p=1) comprises the step of reacting a compound of Formula (V) with a compound of the Formula (VI). For example, one method for preparing carbamoylhydrazide is described in Bull. Soc. Chim. Fr. 1975, 864.

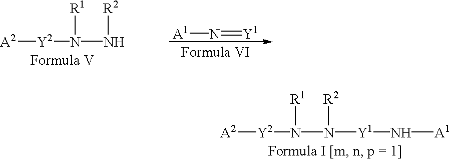

One method for preparing the compounds of Formula (I) (p, m=0 / n=1) comprises the step of reacting a compound of Formula (VII) with a compound of Formula (III). Methods for preparing hydrazide or thiohydrazide are equivalent to the methods for preparing 1,2-diacylhydrazines, 1,2-disulfonylhydrazines or 1-acyl-2-sulfonylhydrazines only that one carbonyl or thiocarbonyl moiety is missing.

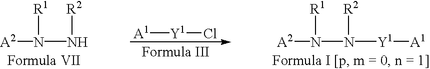

One method for preparing the compounds of Formula (I) (m, n=0 /p=1) comprises the step of reacting a compound of Formula (VIII) with a compound of Formula (VI). Other methods for preparing different ureas are described for example in Organic Synthesis on Solid Phase, Ed. F. Z. Dörwald, p. 331ff, Wiley-VCH, Weinheim, 1999 or in Houben-Weyl, vol. E4, Kohlensäure-Derivate [Carboxylic acid derivatives] Publisher Hagemann, Georg Thieme Verlag, Stuttgart, 1983 and asymmetric ureas are described in R. A. Batey, Tetrahedron Letters 1998, 39, 6267-70. Thioureas for example are described in Bull. Soc. Chim., Belg. Synth. 1978, 87, 229-38, in Org. Synth. 1984, 62, 158-64 or Chem. Rev. 1961, 61, 45-86 J. Comb. Chem., 2000, 2, 75-79 and in Houben-Weyl, Vol. E4, Kohlensäure-Derivate [Carbonic acid derivatives], Editor Hagemann, Georg Thieme Verlag, Stuttgart, 1983, 484-505. Methods to synthezise sulfamides are described in Tetrahedron Letters 1997, Vol. 38, 8691-4 or in WO 01/36383 and guanidine for example are described in J. Parlow et al., J. Org. Chem. 1997, 62, 5908-19.

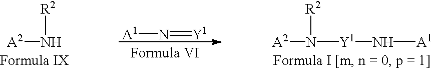

One method for preparing the compounds of Formula (I) (m=0 / n, p=1) comprises the step of reacting a compound of Formula (VII) with a compound of Formula (VI). Possibilities for preparing different semicarbazides or thiosemicarbazides are described by Dobosz et al., Acta Pol. Pharm. 2000, 57, 3, 205-12 or in Indian J. Chem. Sect. B 1999, 38, 9, 1066-9 or in Eur. J. Med. Chem. Chim. Ther. 1999, 34, 2, 153-60 or by Demchenko et. al., Pharm. Chem. J. 1997, 31, 6, 311-3 or Kelarev et al., Russ. J. Org. Chem. 1993, 29, 323-9.

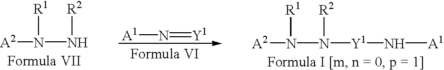

In Formula (I), A¹ or A² each independently represent a C₁-C₁₀-alkyl group which is optionally substituted by one or more substituents R³, or a monocyclic or polycyclic aromatic or non-aromatic ring system which is optionally substituted by one or more substituents R³ and in case of an aromatic ring system contains at least one aromatic ring. The optionally substituted monocyclic or polycyclic aromatic or non-aromatic ring system may also contain one or more groups X selected from S, O, N, NR⁴, SO or SO₂. In preferred embodiments, A¹ and A² each independently represent an optionally substituted C₁-C₂₀-alkyl group or an optionally substituted monocyclic or bicyclic aromatic ring system. In case of substitutions of carbon atoms in the ring system, preferably one, two or three carbon atoms are substituted by a group X, wherein X is selected from the group consisting of S, O, N, NR⁴, SO or SO₂. In one preferred embodiment, one of the carbon atoms is substituted by a group X═O, S, NH.

In Formula (I), A¹ and/or A² independently represent an optionally substituted C₁-C₂₀-alkyl group which is optionally substituted by one or more substituents R³. Preferably A¹ and/or A² independently represent an optionally substituted C₁-C₁₂-alkyl group, said alkyl group may be a straight chain or branched chain alkyl group, and examples include methyl, ethyl, propyl, isopropyl, butyl, t-butyl, isobutyl, pentyl, hexyl, heptyl, octyl, nonyl, decyl, undecyl and dodecyl groups. The term alkyl group also contains alkenyl and alkinyl groups, that means that the alkyl group contains one or more double or triple bounds.

In Formula (I), A¹ and/or A² represent an optionally aromatic or non-aromatic ring system, which is substituted by one or more substituents R³, said ring system may be a phenyl, 1-naphthyl, 2-napthyl, 1-anthracenyl, 2-anthracenyl, 2-pyranyl, 3-pyranyl, 4-pyranyl, 2-thiazolyl, 4-thiazolyl, 5-thiazolyl, 2-oxazolyl, 4-oxazolyl and 5-oxazolyl, in particular 3-pyrazolyl, 4-pyrazolyl, 5-pyrazolyl, 2-pyrazinyl, 3-pyrazinyl, 1-imidazolyl, 2-imidazolyl, 2-thienyl, 3-thienyl, 2-furyl, 3-furyl, 2-pyridyl, 3-pyridyl, 4-pyridyl, benzothiophene, pyrazolo[3,4-b]-pyridyl, 2-pyrimidyl, 4-pyrimidyl and 9H-thioxanthene-10,10-dioxide ring, in which the ring system can be fused to one or more other monocyclic aromatic or non-aromatic rings.

Suitable substituents for A¹ and/or A² are independently H, NO₂, CN, CO₂R⁴, COR⁴, CONR⁴R⁵, NR⁴R⁵, OR⁴, SR⁴, hydroxyalkylamino, hydroxylalkyl, halogen, haloalkyl, haloalkyloxy, SO₂NR⁴R⁵, CO₂NR⁴R⁵, CO₂R⁴, SO₂R⁴, SO₃R⁴, NR⁴R⁵, alkyl, cycloalkyl, arylalkyl, aryl or heteroaryl.

An alkyl group, if not stated otherwise, is preferably a linear or branched chain of 1 to 5 carbon atoms, preferably a methyl, ethyl, propyl, isopropyl, butyl, t-butyl, isobutyl, pentyl or hexyl group, a methyl, ethyl, isopropyl or t-butyl group being most preferred. The alkyl group in the compounds of Formula (I) can optionally be substituted by one or more substituents R³, preferably by halogen.

An cycloalkyl group denotes a non-armoatic ring system containing 4 to 8 carbon atoms, wherein the ring system comprises one or more of the carbon atoms in the ring can be substituted by a group X, X being as defined above.

An alkoxy group denotes an O-alkyl group, the alkyl group being as defined above.

An haloalkyl group denotes an alkyl group which is substituted by one to five preferably three halogen atoms, the alkyl group being as defined above.

A hydroxyalkyl group denotes an HO-alkyl group, the alkyl group being as defined above.

An haloalkyloxy group denotes an alkoxy group which is substituted by one to five preferably three halogen atoms, the alkyl group being as defined above.

A hydroxyalkylamino group denotes an (HO-alkyl)₂-N— group or HO-alkyl-NH— group, the alkyl group being as defined above.

A halogen group is chlorine, bromine, fluorine or iodine, fluorine being preferred.

An aryl group preferably denotes an aromatic group having 5 to 15 carbon atoms, in particular a phenyl group. This aryl group can optionally be substituted by one or more substituents R′, where R³ is as defined above, preferably by haloalkyloxy.

An arylalkyl group denotes an alky group which is substituted by one to three preferably one aryl groups, the alkyl and aryl group being as defined above.

A heteroaryl group denotes a 5- or 6-membered heterocyclic group which contains at least one heteroatom like O, N, S. This heterocyclic group can be fused to another ring. For example, this group can be selected from an oxazol-2-yl, oxazol-4-yl, oxazol-5-yl, thiazol-2-yl, thiazol-4-yl, thiazol-5-yl, isothiazol-3-yl, isothiazol-4-yl, isothiazol-5-yl, 1,2,4-oxadiazol-3-yl, 1,2,4-oxadiazol-5-yl, 1,2,4-thiadiazol-3-yl, 1,2,4-thiadiazol-5-yl, 1,2,5-oxadiazol-3-yl, 1,2,5-oxadiazol-4-yl, 1,2,5-thiadiazol-3-yl, 1-imidazolyl, 2-imidazolyl, 1,2,5-thiadiazol-4-yl, 4-imidazolyl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyrimidinyl, 4-pyrimidinyl, 5-pyrimidinyl, 3-pyridazinyl, 4-pyridazinyl, 2-pyrazinyl, 1-pyrazolyl, 3-pyrazolyl, 4-pyrazolyl, indolyl, indolinyl, benzo-[b]-furanyl, benzo[b]thiophenyl, benzimidazolyl, benzothiazolyl, quinazolinyl, quinoxazolinyl, or preferably isoxazol-3-yl, isoxazol-4-yl, isoxazol-5-yl, quinolinyl, tetrahydroquinolinyl, isoquinolinyl, tetrahydroisoquinolinyl group. This heterocyclic group can optionally be substituted by one or more substituents R³, where R³ is as defined above.

A preferred compound of the present invention is a compound wherein p, m, and n are all 0, A¹ represents a substituted monocyclic aromatic ring system, and A₂ represents an optionally substituted monocyclic aromatic ring system.

A preferred compound of the present invention is a compound wherein p, m and n are all 0, A¹ represents a substituted monocyclic aromatic ring system, and A₂ represents an optionally substituted alkyl group.

A more preferred compound of the present invention is a compound wherein p is 0 and m, n are 1, one of A¹ and A² represent an optionally substituted 5-membered aromatic ring system, and the other one of A¹ and A² represent an optionally substituted alkyl group or a substituted monocyclic aromatic ring system.

A more preferred compound of the present invention is a compound wherein p is 0 and m, n are 1, A¹ and A² represent an optionally substituted 5-membered aromatic ring system.

A more preferred compound of the present invention is a compound wherein p, m, n are all 1, one of A¹ and A² represent an optionally substituted 5-membered aromatic ring system, and the other one of A¹ and A² represent an optionally substituted alkyl group or a substituted monocyclic aromatic ring system.

A more preferred compound of the present invention is a compound wherein p, m, n are all 1, A¹ and A² represents an optionally substituted 5-me(membered aromatic ring system.

A more preferred compound of the present invention is a compound wherein p and n are 1 and m is 0, one of A¹ and A² represent an optionally substituted 5-membered aromatic ring system, and the other one of A¹ and A² represent an optionally substituted alkyl group or a substituted monocyclic aromatic ring system.

A more preferred compound of the present invention is a compound wherein p and n are 1 and m is 0, A¹ and A² represent an optionally substituted 5-membered aromatic ring system.

In the compounds of Formula (I), R is independently H, alkyl, cycloalkyl, aryl or heteroaryl. Preferably, R is H.

In the compounds of Formula (I), R¹ is independently H, alkyl, cycloalkyl, aryl or heteroaryl. Preferably, R¹ is H.

In the compounds of Formula (I), R² is independently H, alkyl, cycloalkyl, aryl or heteroaryl. Preferably, R² is H.

Preferably, R³ in Formula (I) is independently H, halogen, CF₃, OCF₃, phenyl or alkyl.

R⁴ in Formula (I) is independently H, alkyl, cycloalkyl, aryl or heteroaryl. Preferably R⁴ is H.

R⁵ in Formula (I) is independently H, O-alkyl, O-aryl, alkyl, heteroaryl or aryl. Preferably R⁵ is H.

In Formula (I) Y¹ and Y² are independently from each other CO, CS, SO₂ or CNR⁵, preferably both are CO.

In Formula (I) Z is independently S, O, N, NR⁴, CO, CO₂, CS, SO or SO₂. Preferably, Z is O, CO, CO₂.

In Formula (I) X is independently S, O, N, NR⁴, SO or SO₂. Preferably, X is N, S, O, NR⁴.

In Formula (I), most preferably, m and n are 1 and p is 0.

In Formula (I), more preferably, m, n and p are all 0.

In Formula (I), most preferably, m, n and p are all 1.

The compounds of the Formula (I) according to the invention can be also used in form of the corresponding salts with inorganic or organic acids or bases. Examples of such salts are, e.g., alkali metal salts, in particular sodium and potassium salts, or ammonium salts.

In general, the compounds of the present invention can be used to inhibit quorum sensing signaling of bacteria employing HSLs as signal molecules for cell-cell communication. Preferably, the compounds can be applied to the bacteria listed in Table 1, and more preferably to the bacteria of Table 1 that are pathogens. In the following it is explained that the compounds of the present invention can be used as antibacterial agents in various applications.

In a preferred form, the compounds of Formula (I) are useful for the treatment of a variety of human, animal and plant diseases, where bacterial pathogens regulate the expression of virulence genes and other phenotypes, e.g. biofilm formation, through an HSL-based quorum sensing system. Furthermore, as the list of organisms (see Table 1) employing quorum sensing signaling for their virulence continues to increase, the compounds of the invention can be used also for organisms which will be added to the above listed in future.

In a first embodiment, the compounds are useful for the treatment of mammalian in particular human diseases caused by bacteria through the inhibition of the bacterial quorum sensing cascade rendering the pathogen avirulent. Such diseases include endocarditis, respiratory and pulmonary infections (preferably in immunocompromized and cystic fibrosis patients), bacteremia, central nervous system infections, ear infections including external otitis, eye infections, bone and joint infections., urinary tract infections, gastrointestinal infections and skin and soft tissue infections including wound infections, pyoderma and dermatitis which all can be triggered by Pseudomonas aeruginosa. Furthermore, the compounds can be used for the treatment of pulmonary infections caused by Burkholderia cepacia (preferably in immunocompromized and cystic fibrosis patients), gastroenteritis and wound infections caused by Aeromonas hydrophila, sepsis in tropical and subtropical areas caused by Chromobacterium violaceum, diarrhoea with blood and haemolytic uremic syndrome (HUS) caused by Escherichia coli, yersiniosis triggered by Yersinia enterocolitica and Y. pseudotuberculosis, and transfusion-related sepsis and fistulous pyoderma caused by Serratia liquefaciens.

In a second embodiment, the compounds can be used to prevent and/or treat plant diseases, where inhibition of the HSL-mediated signaling system reduces or abolishes virulence of bacterial plant pathogens. Such diseases include crown gall tumors caused by Agrobacterium tumefaciens, soft rot caused by Burkholderia cepacia, Erwinia carotovora and Erwinia chrysanthemi, sweet corn and maize infections caused by Pantoea stewartii and wilt disease caused by Ralstonia solanacearum.

In a third embodiment, the compounds can be used for the prevention and/or treatment of animal diseases, preferably fish diseases such as septicemia caused by Aeromonas hydrophila and Vibrio anguillarum, furunculosis in salmonids caused by Aeromonas salmonicida, prawn infections caused by Vibrio harveyi and enteric redmouth disease caused by Yersinia ruckeri, but also for the prevention and/or treatment of insect diseases caused, for example, by Xenorhabdus nematophilus.

In general, the present invention provides a method for reducing the virulence of bacterial pathogens employing an HSL-based signaling system. In a preferred form, a method is provided to remove, diminish, detach or disperse a bacterial biofilm from a living or nonliving surface by treating the surface with a compound of Formula (I). This method is also useful to prevent biofilm formation on a living or nonliving surface by treating the surface with a compound of Formula (I) before bacterial colonization can initialize. The term “biofilm” refers to cell aggregations comprising either a single type of organism or a mixture of more than one organism, then referred to as “mixed biofilms”. It is clear to persons skilled in the art, that the compounds of the present invention can be applied in a wide variety of different fields such as environmental, industrial and medical applications in order to prevent and/or treat damages or diseases caused by bacteria.

In one aspect, the compounds of Formula (I) can be used for all kinds of surfaces in private and public areas, where it is beneficial to inhibit quorum sensing systems of Gram-negative bacteria in order to prevent and/or treat colonization and biofilm formation. The compound is preferably applied to the surface as a solution of the compound, alone or together with other materials such as conventional surfactants, preferably sodium dodecyl sulfate, or detergents, biocides, fungicides, antibiotics, pH regulators, perfumes, dyes or colorants. In combination with a bacteriocidal agent, e.g., the compounds of Formula (I) inhibit virulence or biofilm formation whilst the bacteriocidal agent kills the pathogens.

In one embodiment, the compounds can be used as antibacterial agent for topical use in cleaning and treatment solutions such as disinfectants, detergents, household cleaner and washing powder formulations in the form of a spray or a dispensable liquid. In a preferred form, these solutions can be applied to windows, floors, clothes, kitchen and bathroom surfaces and other surfaces in the area of food preparation and personal hygiene. In addition, the compounds of Formula (I) can be used as antibacterial ingredients in personal hygiene articles, toiletries and cosmetics such as dentifrices, mouthwashes, soaps, shampoos, shower gels, ointments, creams, lotions, deodorants and disinfectants and storage solutions for contact lenses.

In another embodiment, the compounds can be used to prevent or treat bacterial biofilms in industrial settings such as ship hulls, paper manufacturing, oil recovery, food processing and other applications where process disturbances are referred to biofouling on surfaces. The compounds here can be used in form of a solution, paint or coating. The compounds can also be applied to water processing plants or drinking water distribution systems where the colonized surface (preferably by Pseudomonas aeruginosa) is preferably the inside of an aqueous liquid system such as water pipes, water injection jets, heat exchangers and cooling towers. Until now biocides are the preferred tools to encounter these problems, but since biocides do not have a high specificity for bacteria, they are often toxic to humans as well. This can be circumvented by the application of the compounds of the present invention.

In a further embodiment, the present invention relates to a method of inhibiting and/or preventing medical device-associated bacterial infections. The invention provides articles coated and/or impregnated with a compound of Formula (I) in order to inhibit and/or prevent biofilm formation thereon. The articles are preferably surgical instruments, blood bag systems or medical devices; more preferably either permanently implanted devices such as artificial heart valve, prostethic joint, voice prosthesis, stent, shunt or not permanently implanted devices such as endotracheal or gastrointestinal tube, pacemaker, surgical pin or indwelling catheter.

In a more preferred form, the indwelling catheters are urinary catheters, vascular catheters, peritoneal dialysis catheter, central venous catheters and needleless connectors. The catheter materials can be polyvinylchloride, polyethylene, latex, teflon or similar polymeric materials, but preferably polyurethane and silicone or a mixture thereof. In order to reduce the risk of catheter-related bacterial infections, several catheters coated and/or impregnated with antiseptic or antimicrobial agents such as chlorhexidine/silver-sulfadiazine and minocycline/rifampin, respectively, have been developed. Furthermore, collection bags or layers sandwiched between an external surface sheath and a luminal silicone sheath have been constructed to overcome rapid loss of antimicrobial activity. Nevertheless, the emerging risk of bacterial resistance against traditional antibiotics limits the routine use of antibiotic-coated catheters.

The compounds of the present invention, however, offer the possibility to effectively reduce catheter-related bacterial infections with a low risk of resistance development due to a novel therapeutic strategy targeting highly sensitive signal transduction mechanisms in bacteria. The preferred form of application is the coating and/or impregnating of catheter materials on both the inner and outer catheter surfaces. More preferably, the compounds of Formula (I) can be included in a mixture of antibacterial agents released continously from a catheter-associated depot into the environment.

In a further embodiment, the compounds of the present invention and their pharmacologically acceptable salts can be administered directly to animals, preferably to mammals, and in particular to humans as antibiotics per se, as mixtures with one another or in the form of pharmaceutical preparations which allow enteral or parenteral use and which as active constituent contain an effective dose of at least one compound of the Formula I or a salt thereof, in addition to customary pharmaceutical excipients and additives. The compounds of Formula (I) can also be administered in form of their salts, which are obtainable by reacting the respective compounds with physiologically acceptable acids and bases.

The therapeutics can be administered orally, e.g., in the form of pills, tablets, coated tablets, sugar coated tablets, lozenges, hard and soft gelatin capsules, solutions, syrups, emulsions or suspensions or as aerosol mixtures. Administration, however, can also be carried out rectally, e.g., in the form of suppositories, or parenterally, e.g., in the form of injections or infusions, or percutaneously, e.g., in the form of ointments, creams or tinctures.

In addition to the active compounds of Formula (I), the pharmaceutical composition can contain further customary, usually inert carrier materials or excipients. Thus, the pharmaceutical preparations can also contain additives or adjuvants commonly used in galenic formulations, such as, e.g., fillers, extenders, disintegrants, binders, glidants, wetting agents, stabilizers, emulsifiers, preservatives, sweetening agents, colorants, flavorings or aromatizers, buffer substances, and furthermore solvents or solubilizers or agents for achieving a depot effect, as well as salts for modifying the osmotic pressure, coating agents or antioxidants. They can also contain two or more compounds of the Formula (I) or their pharmacologically acceptable salts and also other therapeutically active substances.

Thus, the compounds of the present invention can be used alone, in combination with other compounds of this invention or in combination with other active compounds, for example with active ingredients already known for the treatment of the afore mentioned diseases, whereby in the latter case a favorable additive effect is noticed. Suitable amounts to be administered to mammalian in particular humans range from 5 to 1000 mg.

To prepare the pharmaceutical preparations, pharmaceutically inert inorganic or organic excipients can be used. To prepare pills, tablets, coated tablets and hard gelatin capsules, e.g., lactose, corn starch or derivatives thereof, talc, stearic acid or its salts, etc. can be used. Excipients for soft gelatin capsules and suppositories are, e.g., fats, waxes, semi-solid and liquid polyols, natural or hardened oils etc. Suitable excipients for the production of solutions and syrups are, e.g., water, alcohol, sucrose, invert sugar, glucose, polyols etc. Suitable excipients for the production of injection solutions are, e.g., water, alcohol, glycerol, polyols or vegetable oils.

The dose can vary within wide limits and is to be suited to the individual conditions in each individual case. For the above uses the appropriate dosage will vary depending on the mode of administration, the particular condition to be treated and the effect desired. In general, however, satisfactory results are achieved at dosage rates of about 0.1 to 100 mg/kg animal body weight preferably 1 to 50 mg/kg. Suitable dosage rates for larger mammals, e.g., humans, are of the order of from about 10 mg to 3 g/day, conveniently administered once, in divided doses 2 to 4 times a day, or in sustained release form.

In general, a daily dose of approximately 0.1 mg to 5000 mg, preferably 10 to 500 mg, per mammalian in particular human individual is appropriate in the case of the oral administration which is the preferred form of administration according to the invention. In the case of other administration forms too, the daily dose is in similar ranges. The compounds of Formula (I) can also be used in the form of a precursor (prodrug) or a suitably modified form, that releases the active compound in vivo.

In a further embodiment, the compounds of the present invention can be used as pharmacologically active components or ingredients of medical devices, instruments and articles with an effective dose of at least one compound of the Formula I or a salt thereof. The amount of the compounds used to coat for example medical device surfaces varies to some extent with the coating method and the application field. In general, however, the concentration range from about 0.01 mg per cm² to about 100 mg per cm². In a similar way the amount of the compounds has to be adjusted to the application mode if the compounds of the invention are used as components or ingredients in cleaning or treatment solutions. In general, effective dosages range from about 0.1 μM to about 1000 mM.

The invention is further illustrated by the following non-limiting Examples, Tables and Figures.

FIG. 1 shows a general N-acyl-L-homoserine lactone structure (R=acyl side chain);

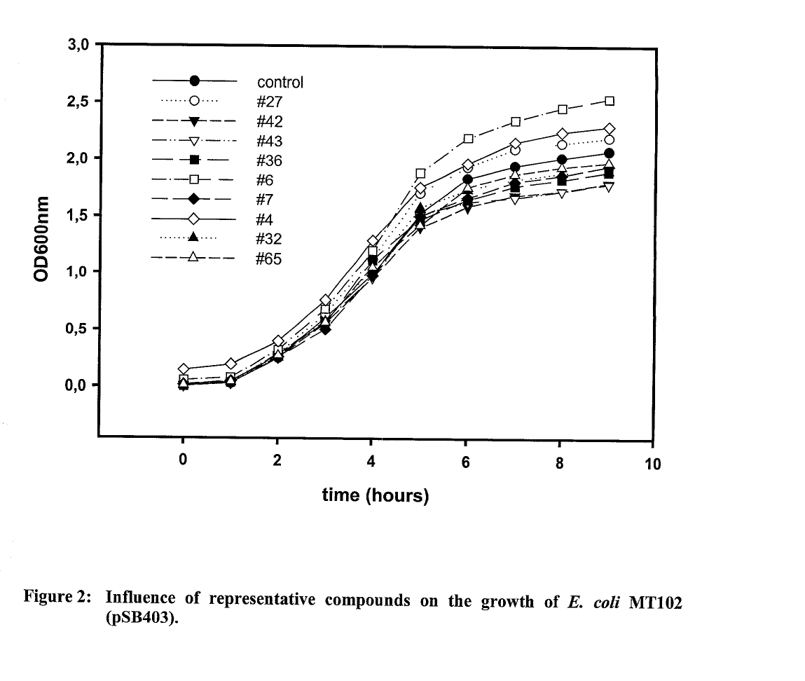

FIG. 2 shows the influence of representative compounds of the invention on the growth of E. coli MT102 (pSB403);

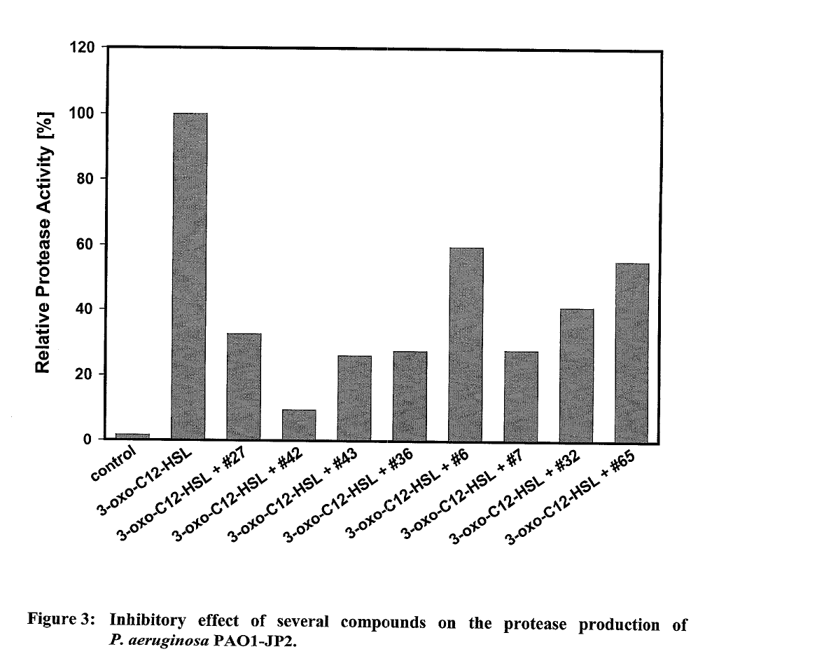

FIG. 3 shows the inhibitory effect of several compounds of the invention on the protease production of P. aeruginosa PAO1-JP2;

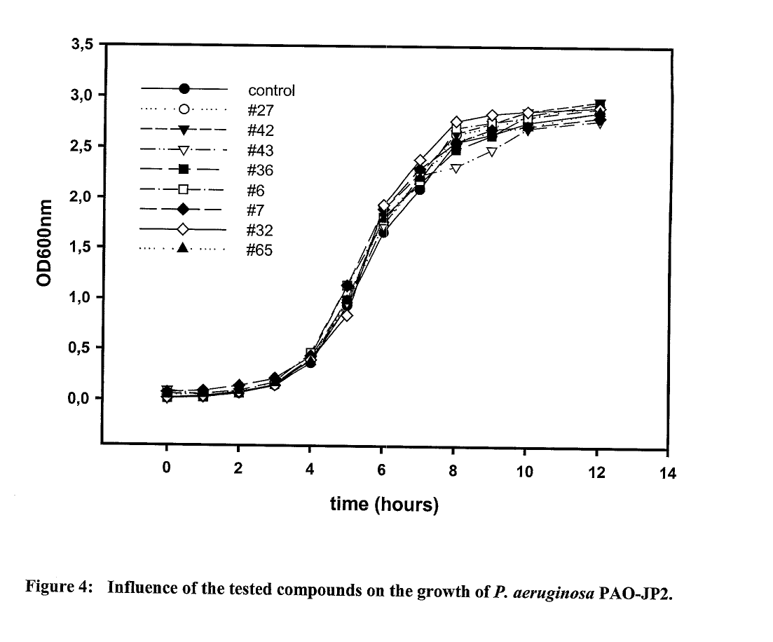

FIG. 4 shows the influence of the tested compounds of the invention on the growth of P. aeruginosa PAO-JP2;

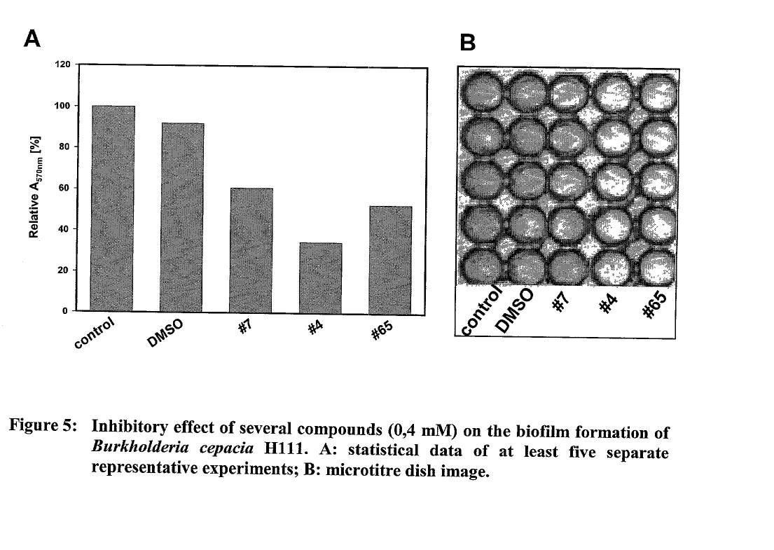

FIG. 5 shows the inhibitory effect of several compounds (test concentation: 0.4 mM) of the invention on the biofilm formation of Burkholderia cepacia H111. FIG. 5A shows the statistical data of at least five separate representative experiments; FIG. 5B shows a microtitre dish image.

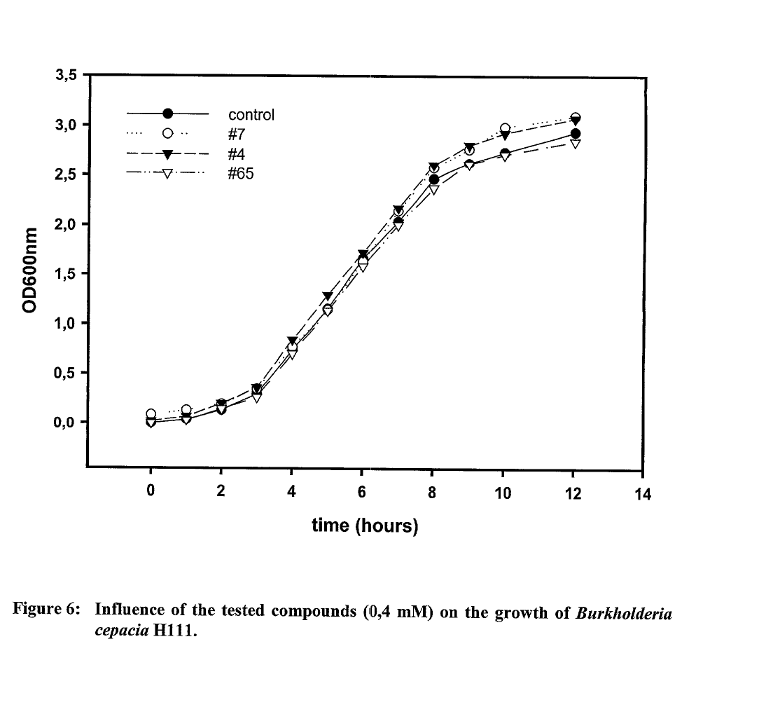

FIG. 6 shows the influence of tested compounds (test concentration: 0.4 mM) of the invention on the growth of Burkholderia cepacia H111.

The following section shows examples for the synthesis of the compounds of the present invention and demonstrate their quorum sensing inhibiting effect.

### EXAMPLES

1. Synthesis of Compounds of Formula (I)

Synthesis Method A (1,2-diacylhydrazine or 1-acyl-2-sulfonylhydrazine derivatives)

A solution of (1.2 eq) acid chloride or (1.2 eq) sulfonyl chloride in tetrahydrofuran was added to a solution of (1 eq) hydrazide in tetrahydrofuran and molecular sieve (0.4 nm) at 0° C. The mixture was stirred at room temperature. After 1 h the reaction mixture was concentrated in vacuum, and the resulting solid was purified by preparative thin layer chromatography (Merck, 20×20 cm, Silica gel 60 F₂₅₄, 1 mm) (CH₂Cl₂:MeOH, 100:1).

Synthesis Method B (1,2-diacylhydrazine or 1-acyl-2-sulfonylhydrazine derivatives)

A solution of (1.2 eq) acid chloride or (1.2 eq) sulfonyl chloride in dimethylformamide was added to a solution of (1 eq) hydrazide in dimethylformamide and (1.2 eq) triethylamine at 0° C. The mixture was stirred at room temperature. After 1 h the reaction mixture was concentrated in vacuum, and the resulting solid was purified by preparative thin layer chromatography (Merck, 20×20 cm, Silica gel 60 F₂₅₄, 1 mm) (CH₂Cl₂:MeOH, 100:1).

Synthesis Method C (1,2-diacylhydrazine or 1-acyl-2-sulfonylhydrazine derivatives)

A solution of (1.2 eq) acid chloride or (1.2 eq) sulfonyl chloride in dichloromethane was added to a solution of (1 eq) hydrazide in dichloromethane and (1.2 eq) triethylamine at 0° C. The mixture was stirred at room temperature. After 1 h the reaction mixture was concentrated in vacuum, and the resulting solid was purified by preparative thin layer chromatography (Merck, 20×20 cm, Silica gel 60 F₂₅₄, 1 mm) (n-hexane:EtOAc, 9:1).

Synthesis Method D (amide derivatives)

A solution of (1.2 eq) acid chloride in tetrahydrofuran was added to a solution of (1 eq) amine in tetrahydrofuran and molecular sieve (0.4 nm) at 0° C. The mixture was stirred at room temperature. After 1 h the reaction mixture was concentrated in vacuum, and the resulting solid was purified by preparative thin layer chromatography (Merck, 20×20 cm, Silica gel 60 F₂₅₄, 1 mm) (CH₂Cl₂:MeOH, 100:1).

Synthesis Method E (amide derivatives)

A solution of (1.2 eq) acid chloride in dichloromethane was added to a solution of (1 eq) amine in dichloromethane and (1.2 eq) triethylamine at 0° C. The mixture was stirred at room temperature. After 1 h the reaction mixture was concentrated in vacuum, and the resulting solid was purified by preparative thin layer chromatography (Merck, 20×20 cm, Silica gel 60 F₂₅₄, 1 mm) (n-hexane:EtOAc, 9:1).

Synthesis Method F (semicarbazide or thiosemicarbazide derivatives)

A solution of (1.3 eq) isocyanate or (1.3 eq) isothiocyanate in tetrahydrofuran was added to a solution of (1 eq) hydrazide in tetrahydrofuran and molecular sieve (0.4 nm) at 0° C. The mixture was stirred at room temperature. After 1 h the reaction mixture was concentrated in vacuum, and the resulting solid was purified by preparative thin layer chromatography (Merck, 20×20 cm, Silica gel 60 F₂₅₄, 1 mm) (CH₂Cl₂:MeOH, 100:5).

Synthesis Method G (hydrazide derivatives)

A solution of (1.2 eq) acid chloride in tetrahydrofuran was added to a solution of (1 eq) hydrazine in tetrahydrofuran and molecular sieve (0.4 nm) at 0° C. The mixture was stirred at room temperature. After 1 h the reaction mixture was concentrated in vacuum, and the resulting solid was purified by preparative thin layer chromatography (Merck, 20×20 cm, Silica gel 60 F₂₅₄, 1 mm) (CH₂Cl₂:MeOH, 100:1).

In the following Table 2, the synthesis method employed in each case for the respective compound or whether the compound was obtained is indicated. Furthermore, the mass found by mass spectrometry, the exact molecular mass, the NMR-data (abbreviations: br.=broad, s=singulet, d=doublet, t=triplet, q=quartet, quint.=quintet, sext.=sextet, m_{c}=multiplet centered, m=multiplet, CHₐᵣ=aromatic H, J=¹H—¹H coupling constant) and the IC₅₀ range as a measure of anti-quorum sensing activity are indicated.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

2. Biosensor Assay

Quorum sensing inhibition of the compounds was investigated with the aid of the bioluminescent sensor strain Escherichia coli MT102 (pSB403) (Winson et al., FEMS Microbiol. Lett. 163:185-92, 1998). Plasmid pSB403 contains the Photobacterium fischeri luxR gene together with the luxI promoter region as a transcriptional fusion to the bioluminescence genes luxCDABE of Photorhabdus luminescence. Although E. coli pSB403 exhibits the highest sensitivity for the Photobacterium fischeri quorum sensing signal N-(3-oxohexanoyl) homoserine lactone (3-oxo-C6-HSL), a wide range of other HSL molecules are detected by the sensor (Winson et al, FEMS Microbiol. Lett. 163:185-92, 1998; Geisenberger et al., FEMS Microbiol. Lett. 184:273-8, 2000).

Inhibitory studies were conducted in a microtitre dish assay as follows: the E. coli sensor strain grown over night in LB medium (Sambrook et al., Molecular Cloning: A Laboratory Maual. 2^{nd} Edn. Cold Spring Harbor Laboratory, New York, 1989) was diluted 1:4 and grown for another 1 hour at 30° C. After addition of 3-oxo-C6-HSL (final concentration 100 nM) 100 μl of an exponential culture suspension were filled in the wells of a FluoroNunc Polysorp microtitre dish. The test compounds were added to the culture in different concentrations and bioluminescence was measured after 4 hours of incubation at 30° C. with a Lamda Fluoro 320 Plus reader (Bio-Tek Instruments). Inhibitor-mediated reduction of light emission was correlated with the value obtained without addition of the test compounds. IC₅₀ values (concentration of inhibitor required for 50% inhibition of the signal compared to the signal without inhibitor) were determined by using a fitting function after drawing a graph of the activities of eight different inhibitor concentrations. The determined IC₅₀ range of each compound is listed in Table 2.

To exclude the possibility that the inhibitory effect is attributed to growth inhibition but not to a specific interaction of the test compound with the sensors quorum sensing system growth curves in the presence and absence of the test compounds were compared. E. coli MT102 (pSB403) was grown in LB medium at 37° C. in the presence of 0.4 mM test compound. Growth was measured as optical density at 600 nm. None of the compounds listed in Table 2 exhibit any growth inhibitory effects on the sensor strain E. coli MT102 (pSB403). FIG. 2 shows the growth curves of representative compounds indicating a specific inhibitory effect of the compounds on the quorum sensing system.

3. Inhibition of Protease Production

The inhibitory effect of the compounds on quorum sensing regulated virulence factors was demonstrated by investigating the expression of extracellular proteases by Pseudomonas aeruginosa. The P. aeruginosa mutant strain PAO-JP2 (Pearson et al., J. Bacteriol. 179:5756-67, 1997) carrying mutations in the quorum sensing genes lasI and rhlI is unable to produce extracellular proteolytic enzymes. Protease expression can be completely restored by external addition of 3-oxo-C12-HSL. The protease assay was performed according to Riedel et al. (J. Bacteriol. 183:1805-9, 2001) with few modifications. PAO-JP2 was grown in LB medium at 30° C. and shaking at 250 rpm to an OD_{600 nm} of 0.5. The test compounds were added at a final concentration of 0.4 mM and the culture was incubated for further 30 min at 30° C. and shaking at 250 rpm. After addition of 3-oxo-C12-HSL at a final concentration of 0.3 μM the cultures were grown for an additional 6 hours at 30° C. and shaking at 250 rpm. The proteolytic activity was measured as described by Ayora & Götz (Mol. Gen. Genet. 242:421-30, 1994). 50 μl culture supernatant were incubated with Azocasein (250 μl 2%, Sigma, St. Louis, Mo.) for 1 hour at 37° C. After precipitation of undigested substrate with trichloroacetic acid (1.2 ml 10%) for 20 minutes at room temperature, followed by 5 minutes centrifugation at 13000 rpm, NaOH (0.75 ml 1M) was added to the supernatant. The relative protease activity was measured as absorbance at 440 nm (OD_{440 nm}) of the supernatant divided by the optical density of the culture (OD_{600 nm}). FIG. 3 demonstrates the inhibitory effect of several compounds on protease production of P. aeruginosa PAO-JP2. The data presented are representative for at least three separate experiments.

To demonstrate that inhibition of protease production is due to a specific interference with the quorum sensing system growth curves in the presence and absence of the test compounds were compared. P. aeruginosa PAO-JP2 was grown in LB medium at 30° C. in the presence of 0.4 mM test compound. Growth was measured as optical density at 600 nm. None of the compounds listed in Table 2 exhibit any growth inhibitory effects on P. aeruginosa PAO-JP2. FIG. 4 shows the growth curves of representative compounds indicating a specific inhibitory effect of the compounds on the quorum sensing system.

4. Inhibition of Biofilm Formation

The bacterial biofilm formation assay was performed in polystyrene microtitre dishes (FluoroNunc Polysorp) according to the method described by O'Toole & Kolter (Mol. Microbiol. 28:449-61, 1998) and Pratt & Kolter (Mol. Microbiol 30:285-93, 1998) with few modifications (Huber et al., Microbiology, 147:25-17-28, 2001). Cells were grown in the wells of the microtitre dishes in 100 μl AB medium (Clark & Maaloe, J. Mol. Biol. 23:99-112, 1967) supplemented with 10 mM sodium citrate (Sigma). After addition of the test compound (0.4 mM) the cells were incubated for 48 hours at 30° C. The medium was then removed and 100 μl of a 1% (w/v) aqueous solution of crystal violet (Merck) was added. Following staining at room temperature for 20 minutes, the dye was removed and the wells were washed thoroughly with water. For quantification of attached cells, the crystal violet was solubilized in a 80:20 (v/v) mixture of ethanol and acetone and the absorbance was determined at 570 nm (Ultrospec Plus spectrometer, Pharmacia). FIGS. 5A and 5B demonstrate the inhibitory effect of several compounds on biofilm formation of Burkholderia cepacia H111 (Römling et al., J. Infect. Dis. 170:1616-21, 1994; Gotschlich et al., Syst. Appl. Microbiol. 24:1-14, 2001). The data presented are representative for at least five separate experiments.

To exclude the possibility that biofilm inhibition is attributed to growth inhibition growth curves in the presence and absence of the test compounds were compared. Burkholderia cepacia H111 was grown in LB medium at 37° C. in the presence of 0.4 mM test compound. Growth was measured as optical density at 600 nm. None of the compounds listed in Table 2 exhibit any growth inhibitory effects on the sensor strain Burkholderia cepacia H111. FIG. 6 shows the growth curves of the tested compounds indicating a specific inhibitory effect of the compounds on the quorum sensing system.

1. Use of compounds of the general Formula (I) 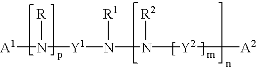
for regulation of the quorum sensing system of microorganisms wherein in Formula (I),
R is H, alkyl, cycloalkyl, aryl or heteroaryl;
R¹ is H, alkyl, cycloalkyl, aryl or heteroaryl;
R² is H, alkyl, cycloalkyl, aryl or heteroaryl;
A¹ an d A² each independently represent an optionally substituted C₁-C₂₀-alkyl group which may contain one or more group(s) Z, or a monocyclic or polycyclic optionally substituted aromatic or non-aromatic ring system which may Contain one or more group(s) X, and in case of a polycyclic ring system, said system contains at least one aromatic ring;
Z is selected from the group consisting of S, O, N, NR⁴, CO, CO₂, CS, SO or SO₂
X is selected from the group consisting of S, O, N, NR⁴, SO or SO₂;
said substituted ring system carries a substituent R³ on one or more of the carbon atoms of said ring system;
said substituted C₁-C₂₀-alkyl group carries a substituent R³ on one or more of the carbon a toms of said alkyl group;
R³ is independently H, OR⁴, SR⁴, hydroxyalkyl, hydroxyalkylamino, cycloalkyl, halogen, haloalkyl, haloalkyloxy, NO₂, CN, SO₂NR⁴R⁵, CO₂NR⁴R⁵, COR⁴, CO₂R⁴, SO₂R⁴, SO₃R⁴, NR⁴R⁵, alkyl, aryl or heteroaryl;
R⁴ is H, alkyl, cycloalkyl, aryl or heteroaryl;
R⁵ is H, O-alkyl, O-aryl, alkyl, heteroaryl or aryl;
Y¹ and Y² are independent from each other C═O, C═S, SO₂ or C═NR⁵;
p is 0, m is 0, n is0;
or p is 0, m is 1, n is 1;
or p is 1, m is 0, n is 1;
or p is 0, m is 0, n is 1;
or p is 1, m is 0, n is 0;
or p is 1, m is 1, n is 1;

2. The use according to claim 1 wherein R¹, R² and R³ are both H, Y¹ and Y² are both C═O, and p is 0, m is 1, n is 1.

3. The use according to claim 1 wherein R¹, R² and R³ are both H, Y¹ and Y² are both C═O, and p is 0, m is 0, n is 0.

4. The use according to claim 1 wherein R¹, R² and R³ are both H, Y¹ and Y² are both C═O, and p is 1, m is 1, n is 1.

5. The use according to any one of claims 1 to 4 as a medicament, antibacterial agent or antifouling coating.

6. The use according to any one of claims 1 to 4 as a medicament, antibacterial agent or antifouling coating for the treatment or prevention of bacterial damages and diseases.

7. The use according to any one of claims 1 to 4 as a medicament, antibacterial agent or antifouling coating for the treatment or prevention of bacterial damages and diseases caused by Gram-negative bacteria.

8. The use according to any one of claims 1 to 4 as a medicament, antibacterial agent or antifouling coating for the treatment or prevention of bacterial damages and diseases caused by Pseudomonas aeruginosa or Burkholderia cepacia.

9. The use according to any one of claims 1 to 4 wherein the quorum sensing system of microorganisms is blocked.

10. The use according to claim 5 wherein the quorum sensing system of microorganisms is blocked.

11. The use according to claim 6 wherein the quorum sensing system of microorganisms is blocked.

12. The use according to claim 7 wherein the quorum sensing system of microorganisms is blocked.

13. The use according to claim 8 wherein the quorum sensing system of microorganisms is blocked.

14. The use according to any one of claims 1 to 4 wherein the expression of quorum sensing dependent virulence factors is blocked.

15. The use according to claim 5 wherein the expression of quorum sensing dependent virulence factors is blocked.

16. The use according to claim 6 wherein the expression of quorum sensing dependent virulence factors is blocked.

17. The use according to claim 7 wherein the expression of quorum sensing dependent virulence factors is blocked.

18. The use according to claim 8 wherein the expression of quorum sensing dependent virulence factors is blocked.

19. The use according to claim 9 wherein the expression of quorum sensing dependent virulence factors is blocked.

20. The use according to any one of claims 1 to 4 for the treatment of biofilms or for inhibiting biofilm formation.

21. The use according to claim 5 for the treatment of biofilms or for inhibiting biofilm formation.

22. The use according to claim 6 for the treatment of biofilms or for inhibiting biofilm formation.

23. The use according to claim 7 for the treatment of biofilms or for inhibiting biofilm formation.

24. The use according to claim 8 for the treatment of biofilms or for inhibiting biofilm formation.

25. The use according to claim 9 for the treatment of biofilms or for inhibiting biofilm formation.

26. The use according to any one of claims 1 to 4 for the treatment of biofilms or for inhibiting biofilm formation on medical articles, instruments and devices.

27. The use according to claim 5 for the treatment of biofilms or for inhibiting biofilm formation on medical articles, instruments and devices.

28. The use according to claim 6 for the treatment of biofilms or for inhibiting biofilm formation on medical articles, instruments and devices.

29. The use according to claim 7 for the treatment of biofilms or for inhibiting biofilm formation on medical articles, instruments and devices.

30. The use according to claim 8 for the treatment of biofilms or for inhibiting biofilm formation on medical articles, instruments and devices.

31. The use according to claim 9 for the treatment of biofilms or for inhibiting biofilm formation on medical articles, instruments and devices.

32. The use according to any one of claims 1 to 4 for the treatment of biofilms or for inhibiting biofilm formation in disinfectants, cleaning and treatment solutions.

33. The use according to claim 5 for the treatment of biofilms or for inhibiting biofilm formation in disinfectants, cleaning and treatment solutions.

34. The use according to claim 6 for the treatment of biofilms or for inhibiting biofilm formation in disinfectants, cleaning and treatment solutions.

35. The use according to claim 7 for the treatment of biofilms or for inhibiting biofilm formation in disinfectants, cleaning and treatment solutions.

36. The use according to claim 8 for the treatment of biofilms or for inhibiting biofilm formation in disinfectants, cleaning and treatment solutions.

37. The use according to claim 9 for the treatment of biofilms or for inhibiting biofilm formation in disinfectants, cleaning and treatment solutions.

38. The use according to any one of claims 1 to 4 for the treatment of biofilms or for inhibiting biofilm formation in personal hygiene articles, toileteries and cosmetics.

39. The use according to claim 5 for the treatment of biofilms or for inhibiting biofilm formation in personal hygiene articles, toileteries and cosmetics.

40. The use according to claim 6 for the treatment of biofilms or for inhibiting biofilm formation in personal hygiene articles, toileteries and cosmetics.

41. The use according to claim 7 for the treatment of biofilms or for inhibiting biofilm formation in personal hygiene articles, toileteries and cosmetics.

42. The use according to claim 8 for the treatment of biofilms or for inhibiting biofilm formation in personal hygiene articles, toileteries and cosmetics.

43. The use according to claim 9 for the treatment of biofilms or for inhibiting biofilm formation in personal hygiene articles, toileteries and cosmetics.

44. The use according to any one of claims 1 to 4 for the treatment of biofilms or for inhibiting biofilm formation in personal hygiene articles, toileteries and cosmetics.

45. The use according to claim 5 for the treatment of biofilms or for inhibiting biofilm formation in personal hygiene articles, toileteries and cosmetics.

46. The use according to claim 6 for the treatment of biofilms or for inhibiting biofilm formation in personal hygiene articles, toileteries and cosmetics.

47. The use according to claim 7 for the treatment of biofilms or for inhibiting biofilm formation in personal hygiene articles, toileteries and cosmetics.

48. The use according to claim 8 for the treatment of biofilms or for inhibiting biofilm formation in personal hygiene articles, toileteries and cosmetics.

49. The use according to claim 9 for the treatment of biofilms or for inhibiting biofilm formation in personal hygiene articles, toileteries and cosmetics.

50. The use according to any one of claims 1 to 4 for the treatment of biofilms or for inhibiting biofilm formation in industrial settings.

51. The use according to claim 5 for the treatment of biofilms or for inhibiting biofilm formation in industrial settings.

52. The use according to claim 6 for the treatment of biofilms or for inhibiting biofilm formation in industrial settings.

53. The use according to claim 7 for the treatment of biofilms or for inhibiting biofilm formation in industrial settings.

54. The use according to claim 8 for the treatment of biofilms or for inhibiting biofilm formation in industrial settings.

55. The use according to claim 9 for the treatment of biofilms or for inhibiting biofilm formation in industrial settings.

56. The use according to any one of claims 1 to 4 for the treatment of biofilms or for inhibiting biofilm formation in industrial settings wherein the industrial setting is selected from the group consisting of ship hulls, food processing systems, oil recovery or paper manufacturing plants.

57. The use according to claim 5 for the treatment of biofilms or for inhibiting biofilm formation in industrial settings wherein the industrial setting is selected from the group consisting of ship hulls, food processing systems, oil recovery or paper manufacturing plants.

58. The use according to claim 6 for the treatment of biofilms or for inhibiting biofilm formation in industrial settings wherein the industrial setting is selected from the group consisting of ship hulls, food processing systems, oil recovery or paper manufacturing plants.

59. The use according to claim 7 for the treatment of biofilms or for inhibiting biofilm formation in industrial settings wherein the industrial setting is selected from the group consisting of ship hulls, food processing systems, oil recovery or paper manufacturing plants.

60. The use according to claim 8 for the treatment of biofilms or for inhibiting biofilm formation in industrial settings wherein the industrial setting is selected from the group consisting of ship hulls, food processing systems, oil recovery or paper manufacturing plants.

61. The use according to claim 9 for the treatment of biofilms or for inhibiting biofilm formation in industrial settings wherein the industrial setting is selected from the group consisting of ship hulls, food processing systems, oil recovery or paper manufacturing plants.

62. The use according to any one of claims 1 to 4 for the treatment of biofilms or for inhibiting biofilm formation in environmental settings.

63. The use according to claim 5 for the treatment of biofilms or for inhibiting biofilm formation in environmental settings.

64. The use according to claim 6 for the treatment of biofilms or for inhibiting biofilm formation in environmental settings.

65. The use according to claim 7 for the treatment of biofilms or for inhibiting biofilm formation in environmental settings.

66. The use according to claim 8 for the treatment of biofilms or for inhibiting biofilm formation in environmental settings.

67. The use according to claim 9 for the treatment of biofilms or for inhibiting biofilm formation in environmental settings.

68. The use according to any one of claims 1 to 4 for the treatment of biofilms or for inhibiting biofilm formation in environmental settings wherein the environmental setting is selected from the group consisting of water distribution or cooling water systems.

69. The use according to claim 5 for the treatment of biofilms or for inhibiting biofilm formation in environmental settings wherein the environmental setting is selected from the group consisting of water distribution or cooling water systems.

70. The use according to claim 6 for the treatment of biofilms or for inhibiting biofilm formation in environmental settings wherein the environmental setting is selected from the group consisting of water distribution or cooling water systems.

71. The use according to claim 7 for the treatment of biofilms or for inhibiting biofilm formation in environmental settings wherein the environmental setting is selected from the group consisting of water distribution or cooling water systems.

72. The use according to claim 8 for the treatment of biofilms or for inhibiting biofilm formation in environmental settings wherein the environmental setting is selected from the group consisting of water distribution or cooling water systems.

73. The use according to claim 9 for the treatment of biofilms or for inhibiting biofilm formation in environmental settings wherein the environmental setting is selected from the group consisting of water distribution or cooling water systems.
